(19) United States
(12) Patent Application Publication
(10) Pub. No.: US 2022/0112237 A1
(43) Pub. Date: Apr. 14, 2022

(54) AMINO ACID COMPOSITION HAVING CO-AMORPHOUS STRUCTURE

(71) Applicant: AJINOMOTO CO., INC., Tokyo (JP)

(72) Inventor: Jun Sen, Kanagawa (JP)

(73) Assignee: AJINOMOTO CO., INC., Tokyo (JP)

(21) Appl. No.: 17/559,470

(22) Filed: Dec. 22, 2021

Related U.S. Application Data
(63) Continuation of application No. PCT/JP2020/024796, filed on Jun. 24, 2020.

(30) Foreign Application Priority Data
Jun. 25, 2019 (JP) 2019-117131

(51) Int. Cl. C07K 5/072; B02C 17/18; B02C 19/00

(52) U.S. Cl. CPC C07K 5/06095; A23L 33/175; B02C 19/0056; B02C 17/1815

(57) ABSTRACT

The present invention provides a means of raising the solubility of even a slightly soluble amino acid, without needing an expensive peptide or varying the pH. The present invention also provides a composition containing at least two amino acids which are formed into a co-amorphous structure.

This application is a Continuation of, and claims priority under 35 U.S.C. § 120 to, International Application No. PCT/JP2020/024796, filed Jun. 24, 2020, and claims priority therethrough under 35 U.S.C. § 119 to Japanese Patent Application No. 2019-117131, filed Jun. 25, 2019, the entireties of which are incorporated by reference herein.

### BACKGROUND

### Technical Field

This invention relates to an amino acid composition having a co-amorphous structure and a method of producing it.

### Background Art

Among foods and beverages supplemented with amino acids, and particularly those that are dissolved in water prior to ingesting, as well as high-calorie transfusion solutions containing amino acids, various devices have been made in order to dissolve slightly soluble amino acids to a desired concentration.

JP 7-64741 B discloses highly soluble dipeptides containing a desired amino acid to be administered, such as L-aspartyl-leucine and L-aspartyl-L-tyrosine. The dipeptides are hydrolyzed into single amino acids and absorbed internally.

JP No. 3368897 discloses solutions produced by dissolving 3 types of branched chain amino acids composed of isoleucine, leucine, and valine in water at a prescribed ratio at pH 4.5-2.2 in the presence of an organic acid and/or inorganic acid.

### SUMMARY

The cost of manufacturing peptides made up of amino acids is higher than manufacturing the component amino acids. The method of adjusting the pH using an organic acid is sometimes restricted because it cannot be added to a product containing an unstable compound at the pH range, or because only a slightly soluble amino acid must be dissolved separately.

An aspect of the invention is to provide a means of raising the solubility of an amino acid without using an expensive peptide or varying the pH. Described herein is a method of combining two or more amino acids so that they are amorphous, resulting in a co-amorphous structure of amino acids that is stably maintained.

Namely, it is an aspect of present invention to provide the composition of amino acids comprising amino acid 1 and amino acid 2, wherein at least amino acid 1 and amino acid 2 form a co-amorphous structure, wherein said amino acid 1 is represented by R₁—CH (NH₂) COOH, and wherein said amino acid 1 is selected from the group consisting of: A) a basic amino acid wherein R₁ contains an amino group or imide bond, B) a sulfur-containing amino acid wherein R₁ contains a sulfide bond, a disulfide bond, or a thiol group, and C) tyrosine; and wherein said amino acid 2 is represented by R₂—CH (NH₂) COOH; wherein when the amino acid 1 is basic, then amino acid 2 is selected from the group consisting of: a) a sulfur-containing amino acid wherein R₂ contains a sulfide bond, a disulfide bond, or a thiol group, b) alanine, c) valine, e) leucine, f) isoleucine, g) tyrosine, h) serine, and i) γ-aminobutyric acid; wherein when the amino acid 1 is the sulfur-containing amino acid, the amino acid 2 is selected from the group consisting of: i) a basic amino acid wherein R₂ is an amino group or imide bond, ii) alanine, iii) valine, iv) leucine, v) isoleucine, vi) tyrosine, vii) serine, viii) glutamic acid or a salt thereof, ix) hydroxyproline, and x) γ-aminobutyric acid; wherein when the amino acid 1 is tyrosine, the amino acid 2 is serine.

It is further aspect of the present invention to provide the composition as described above, which consists essentially of the amino acid 1 and the amino acid 2.

It is further aspect of the present invention to provide the composition as described above, wherein said amino acid 1 is selected from the group consisting of lysine, arginine, histidine, and salts thereof; and wherein said amino acid 2 is selected from the group consisting of cystine, cysteine, methionine, alanine, valine, leucine, isoleucine, tyrosine, serine, and γ-aminobutyric acid.

It is further aspect of the present invention to provide the composition as described above, wherein said amino acid 1 is cystine, cysteine, methionine, and wherein said amino acid 2 is lysine, arginine histidine, alanine, valine, leucine, isoleucine, tyrosine, serine, glutamic acid, salts thereof, hydroxyproline, and γ-aminobutyric acid.

It is further aspect of the present invention to provide the composition as described above, wherein said amino acid 2 is cystine or tyrosine.

It is further aspect of the present invention to provide the composition as described above, wherein said amino acid 2 is serine or γ-aminobutyric acid.

It is further aspect of the present invention to provide the composition as described above, wherein the amino acid 1 and the amino acid 2 are selected from the group consisting of tyrosine and arginine, tyrosine and serine, tyrosine and lysine hydrochloride, tyrosine and histidine, cystine and arginine, cystine and serine, cystine and sodium glutamate monohydrate, cystine and hydroxyproline, cystine and histidine, cystine and γ-aminobutyric acid, leucine and arginine, leucine and lysine hydrochloride, leucine and histidine, leucine and cystine, isoleucine and arginine, isoleucine and lysine hydrochloride, isoleucine and histidine, isoleucine and cystine, valine and arginine, valine and lysine hydrochloride, valine and histidine, valine and cystine, methionine and arginine, and methionine and lysine hydrochloride.

It is further aspect of the present invention to provide the composition as described above, wherein the amino acid 1 and the amino acid 2 are selected from the group consisting of tyrosine and arginine, tyrosine and serine, tyrosine and lysine hydrochloride, tyrosine and histidine, cystine and arginine, cystine and lysine hydrochloride, cystine and serine, cystine and sodium glutamate monohydrate, cystine and hydroxyproline, cystine and histidine, cystine and γ-aminobutyric acid, leucine and arginine, isoleucine and arginine, valine and arginine, methionine and arginine, and methionine and lysine hydrochloride

It is further aspect of the present invention to provide the composition as described above, wherein the amino acid 1 is tyrosine, and the amino acid 2 is lysine hydrochloride, serine, or histidine.

It is further aspect of the present invention to provide the composition as described above, wherein the amino acid 1 is cystine, and the amino acid 2 is serine, lysine hydrochloride, or γ-aminobutyric acid.

It is further aspect of the present invention to provide the composition as described above, wherein the amino acid 1 is arginine, and the amino acid 2 is valine, leucine, or isoleucine.

It is further aspect of the present invention to provide the composition as described above, wherein the amino acid 1 is lysine hydrochloride, and the amino acid 2 is tyrosine, cystine or methionine.

It is further aspect of the present invention to provide the composition as described above, wherein the ratio of amino acid 1 to amino acid 2 in the composition is a molar ratio of 0.1-10.

It is further aspect of the present invention to provide the composition as described above, wherein the ratio of amino acid 1 to amino acid 2 is a molar ratio of 6:4-4:4.

It is aspect of the present invention to provide a beverage containing the composition as described above.

It is aspect of the present invention to provide a medicine containing the composition as described above.

It is aspect of the present invention to provide an aromatic or a cosmetic containing the composition as described above.

It is aspect of the present invention to provide a method of producing the composition as described above, which comprises a) placing at least two amino acids into a ball mill at a molar ratio of 1:0.1-10, and adding a pulverizing assistant at 0.1-5 weight % relative to the total amount of all amino acids, and b) driving the ball mill at a rotational speed of 200-1200 rpm for 15 minutes to 12 hours.

It is an aspect of the present invention to provide the method as described above, wherein the ball mill is cooled to 5-15° C. during the driving of the ball mill.

Generally, amino acid crystals have an inherent lattice energy and solubility. However, since the co-amorphous structure is not a crystal, the lattice energy can be lowered to increase the amount that is dissolved. Therefore, the amount of the co-amorphous structure dissolved can be increased.

The amino acids employed in the invention generally exist in the form of crystal. The amorphous state of single amino acids is unstable, and such instability transfers to crystals. In crystals, molecules are present in an ordered arrangement to form a crystal lattice. When in an amorphous state, there is no regular order such as a crystal lattice, and interactions which stabilize the solid state such as lattice energy, are very few and small. Therefore, molecules disperse and hydrate easily, compared with crystals, and are able to dissolve very quickly.

Although the present invention is not restrained by any theory, a co-amorphous structure is structurally unstable compared to crystals, and co-amorphous structures are easily soluble due to low lattice energy. This is recognized generally in the medical field, where the solubility of crystal is called kinetic solubility and the solubility of amorphous structure is called thermodynamic solubility. Furthermore, the reason why the co-amorphous structures are stable in a supernatant having an amino acid concentration that is higher than that of crystals, is that a long time is required for the precipitation (falling out of solution) of the amino acid crystals from the co-amorphous structure compared with crystal. When crystals are dissolved in water, an associate holding crystal structure is formed therein. While, since a co-amorphous structure has no crystal structure, a completely random association is formed in water. The random association is necessary for a crystal structure to form, and this transformation to a crystal structure can take a long time.

Incidentally, regarding amino acid, solid solutions, for example, that of three amino acids of valine, leucine, and isoleucine are known (WO 2010/050168), and it is also known that the dissolving speed of valine, leucine, and isoleucine in the solid solution are improved. However, the solid solution has a structure where a part of the crystal lattice is replaced by another molecule, or where another molecule enters into the space of crystal lattice. Namely, the solid solution is a crystal, and does not have a random structure like an amorphous structure. The dissolving speed of solid solution is considerably slower than the co-amorphous structure of the invention.

The amino acid mixture of the invention is highly stable, and solubility of the mixture is increased without using an expensive peptide, and even slightly soluble amino acids can be dissolved easily. Moreover, since an amorphous structure having high energy can be maintained stably, there is a merit, such as to raise absorbability of the amino acid or to develop a new use.

### BRIEF DESCRIPTION OF THE DRAWINGS

FIG. 1 is a drawing of powder X-ray spectra showing that Tyr-Arg is co-amorphous.

FIG. 2 is a graph showing the results of a dissolution test of Tyr-Arg co-amorphous material.

FIG. 3 is a drawing of powder X-ray spectra obtained in a preservation stability test of Tyr-Arg co-amorphous material.

FIG. 4 is a drawing of powder X-ray spectra of Tyr and various amino acids.

FIG. 5 is a graph showing the results of a dissolution test of Tyr-LysH co-amorphous material and Tyr crystal.

FIG. 6 is a drawing of powder X-ray spectra of Cys2-Arg and simplex Cys2.

FIG. 7 is a drawing of powder X-ray spectra of combinations of Cys2 and various amino acids.

FIG. 8 is a drawing of powder X-ray spectra of combinations of Leu, etc. and various other amino acids.

FIG. 9 is a drawing of powder X-ray spectra of Met-LysH, Met-Arg, and simplex Met.

FIG. 10 is a drawing of powder X-ray spectra of Tyr-Arg in which the pulverization time was changed.

FIG. 11 is a drawing of powder X-ray spectra of Tyr-Arg and simplex Tyr produced by a spray dryer.

FIG. 12 is a drawing of powder X-ray spectra of Tyr-Arg and Leu-Arg produced by a lyophilizer.

### DETAILED DESCRIPTION OF EXEMPLARY EMBODIMENTS

The amino acids forming the co-amorphous structure as described herein are not limited to α-amino acids, but may also be β-amino acids, such as β-alanine; and γ-amino acids, such as γ-aminobutyric acid, δ-amino acids, or the like.

However, the co-amorphous structure is generally composed of amino acid 1 and amino acid 2. The amino acid 1 is represented by R₁—CH (NH₂) COOH, the amino acid 2 is represented by R₂—CH (NH₂) COOH, and both are α-amino acids. R₁ and R₂ can be one of, respectively, a hydrogen group, alkyl groups having a 1-5 carbons, and groups containing at least one of a hydroxyl group, amino group, carboxyl group, aromatic ring, sulfide bond, disulfide bond, thiol group, and/or a ring structure.

The amino acid where R₁ or R₂ is a hydrogen group is glycine, and the amino acids where R₁ or R₂ is an alkyl group having 1-5 carbons are alanine, valine, leucine, isoleucine, or the like. The amino acids containing a hydroxyl group are serine, threonine, or the like; the amino acids containing an amino group are lysine, arginine, ornithine, glutamine, asparagine, or the like; and the amino acids containing a carboxyl group are aspartic acid, glutamic acid, or the like. The amino acids containing an aromatic ring are phenylalanine, tyrosine, or the like; the amino acids containing a sulfide or disulfide bond are methionine, cystine, or the like; and the amino acid containing a thiol group is cysteine or the like. The amino acids containing a ring structure are histidine, tryptophan, proline, hydroxyproline, or the like; which contains a heterocyclic ring. The amino acid 1 and amino acid 2 can also include imino acids, such as proline and hydroxyproline.

These amino acids have a tendency to form the co-amorphous structure, and it is preferable that at least one of the amino acids is able to form the co-amorphous structure. The amino acids able to form the co-amorphous structure are basic amino acids containing an amino group or imide bond in R₁ or R₂, such as lysine, arginine, ornithine, histidine, and their salts; and sulfur-containing amino acids containing sulfide or a disulfide bond or a thiol group in R₁ or R₂, such as cysteine, cystine, and methionine.

An aspect as described herein is to be able to dissolve slightly soluble amino acids easily, and the slightly soluble amino acids are cystine, tyrosine, phenylalanine, leucine, isoleucine, tryptophan, valine, methionine, and the like. The amino acid that can be combined with the slightly soluble amino acid may be any one capable of forming the co-amorphous structure, and is not particularly limited. For example, it may be also a slightly soluble amino acid. Examples of the amino acid being combined with the slightly soluble amino acid are alanine, valine, leucine, isoleucine, lysine, arginine, histidine, ornithine, glutamine, asparagine, aspartic acid, glutamic acid, serine and the like; and, arginine, lysine, histidine, and cystine are particular examples.

Particularly exemplary combinations are tyrosine and arginine, tyrosine and serine, tyrosine and lysine hydrochloride, tyrosine and histidine, cystine and arginine, cystine and serine, cystine and sodium glutamate monohydrate, cystine and hydroxyproline, cystine and histidine, cystine and γ-aminobutyric acid, leucine and arginine, leucine and lysine hydrochloride, leucine and histidine, leucine and cystine, isoleucine and arginine, isoleucine and lysine hydrochloride, isoleucine and histidine, isoleucine and cystine, valine and arginine, valine and lysine hydrochloride, valine and histidine, valine and cystine, methionine and arginine, and methionine and lysine hydrochloride.

The combination of the amino acids can be determined depending on the intended use, and can be combinations of amino acids which have been previously used in combination. Moreover, the composition of amino acids is not limited to two types of amino acids, and may include three or more amino acids, which form co-amorphous structure.

The mixing ratio of the amino acids in the amino acid composition having a co-amorphous structure may be any ratio capable of forming the co-amorphous structure, and for example, in the case of two amino acids, the mixing ratio may be a molar ratio of 1:0.1 to 1:10, 1:0.2 to 1:5, 1:0.5 to 1:2, 1:0.6 to 1:2, 6:4 to 4:6, or 1:1.

The co-amorphous structure includes structures wherein all of the component amino acids maintain a stable co-amorphous state, and can be in a state where each amino acid is dispersed and mixed uniformly. Every amino acid is in a solid state, and the degree of dispersion varies according to the preparation. Namely, when a method of dissolving and solidifying the component amino acids is utilized, such as spray drying, melt quenching (hot-melt), or lyophilization, each amino acid becomes in a solidified state in the solution, namely in a mixed state at a unit of molecule. While, when a method of mixing and grinding each amino acid powder is utilized, such as grinding by a ball mill, the mixture is in a state of mixed fine powders. Even in such a case, the respective amino acid molecules become capable of interacting with each other, and they are rendered to very fine state (about 5 to 30 μm in median diameter).

Whether each component amino acid in the composition is incorporated into the co-amorphous structure or not can be confirmed by a known method, such as X-ray diffractometry or Raman spectroscopy.

The composition may have at least two amino acids that form the co-amorphous structure, and may also include three or more amino acids, including amino acid(s) other than those which form the co-amorphous structure. In addition, the composition having a co-amorphous structure may include other amino acid(s) that do not form the co-amorphous structure, or components other than amino acid(s) which form or do not form the co-amorphous structure, or a salt of the amino acid(s) or other components.

The co-amorphous structure of amino acid can be produced by grinding using a ball mill, spray drying, melt quenching, lyophilizing, or the like.

As the ball mill, for example, “Ball mill Emax”, manufactured by Verder Scientific Co., Ltd. can be used. The method of using the ball mill may be carried out by putting the amino acids into the ball mill at a prescribed mixing ratio, adding a pulverizing assistant, if necessary, and grinding to mix them until the co-amorphous structure is formed. The amino acids may be either crystal or amorphous material, and typically, they are crystal. The pulverizing assistant is added in order to prevent concretion, and it is usually sufficient that ethanol or the like is added in an amount of about 0.1 to 5% by weight, or 1 to 3% by weight relative to the total amino acids. Pulverization conditions are set so that the crystal lattice of each amino acid collapses and at least two amino acids form the co-amorphous structure, and may be, generally, at a rotary speed of the ball mill of 200 to 1200 rpm for 15 minutes to 12 hours, at 600 to 1200 rpm for 8 to 12 hours, or at 800 to 1200 rpm for 12 hours or more. During pulverizing, since the temperature of the ball mill increases, it is desirable to cool the pot of the ball mill to 15° C. or less, preferably 5 to 12° C. The pulverizing assistant, such as ethanol, is volatilized while pulverizing, and does not remain in the mixture. As the ball mill, Planetary B all Mill (manufactured by Kurimoto, Ltd.), Attritor (manufactured by Nippon Coke & Engineering Co., Ltd.), or surface modification apparatus (Simoloyer, manufactured by Zoz GmbH) can be used.

When producing by spray drying, an aqueous solution containing each amino acid at a prescribed ratio is prepared, and it is spray-dried at a temperature, such as 160° C. or more. It is desirable to evaporate the water prior to forming crystal lattice.

The composition of amino acids formed into a co-amorphous structure can be utilized widely for foods and drinks, for example, as drips in supplements and health foods, as well as used in medicines, perfumes, cosmetics, and the like. Particularly, it can be used to adjust the pH of a salt with an organic acid, or to substitute for a drip in a supplement where an amino acid is made into a dipeptide, such as L-aspartyl-L-leucine or L-aspartyl-L-tyrosine. The amino acids to be combined can be selected from the amino acids employed in each use.

### EXAMPLES

### Example 1

(1) Preparation of Tyr•Arg Co-Amorphous Mixture

5.1 g (0.028 mol) of tyrosine (Tyr) (manufactured by Ajinomoto Co., Inc.), 4.9 g (0.028 mol) of arginine (Arg) (manufactured by Ajinomoto Co., Inc.) and as the pulverizing assistant, 0.1 mL of ethanol, were placed in a 125 mL pot made of zirconia. Fifty 10 mm Φ balls made of zirconia were put in the pot and set in a ball mill (“Retsch Emax”, manufactured by Verder Scientific Co., Ltd.). The amino acids were ground and mixed at a rotary speed of 1,000 rpm at about 28° C. for 12 hours, while cooling water at 15° C. was streamed into the outer periphery of the pot, and 9.5 g powder was obtained.

(2) Confirmation of Formation of Amorphous Product

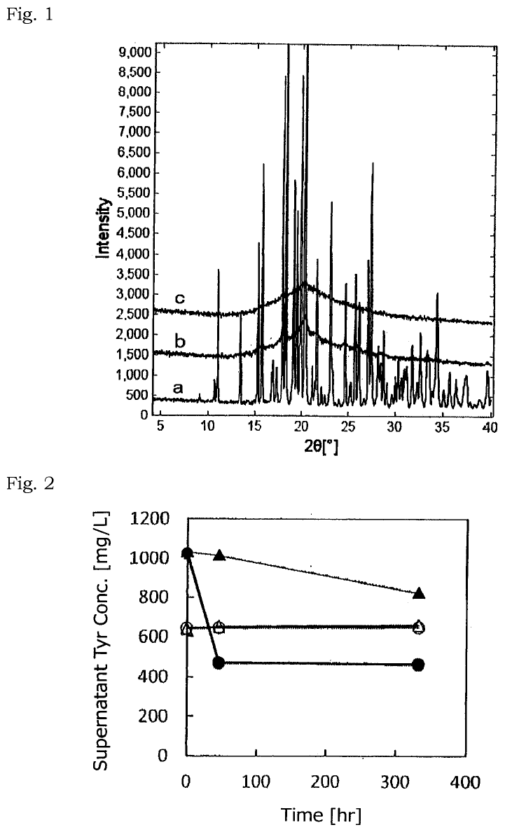

The solid powder obtained in (1) was measured by the powder X-ray diffraction apparatus (“Empyrean”, manufactured by Malvern Panalytical), and the powder X-ray diffraction patterns of the solid were obtained. The results are shown in FIG. 1. In the drawing, line a represents the pattern of the mixture at time zero, line b represents time zero plus 4 hours, and line c represents time zero plus 12 hours. As a result, a well-defined peak characteristic of crystal structure was not found. From this, it was confirmed that the solid obtained in (1) was amorphous.

(3) Test to Confirm the Dissolved Amount

Into four 200 mL sample bottles made of glass (A, B, C, D), 100 mL of ultra-pure water was placed in each one, and 138 mg, 138 mg, 220 mg, 220 mg of the solid obtained in (1) was placed in each bottle, respectively, in order beginning with A, and stirred by using a magnetic stirrer (“B-1 Magnetic Stirrer Octopus”, manufactured by As One Corporation). After the powder was dissolved completely, the pH was adjusted using 5 mol/L hydrochloric acid to be within the range of 6.5-7.5. Bottles A-D were immersed in a water bath (“NCG-3300”, manufactured by Tokyo Rikakikai Co., Ltd.) at 25° C., and bottles A and C were stirred by the magnetic stirrer, and bottles B and D were allowed to stand without stirring. After 2 hours, 2 mL was sampled from each bottle, and filtered using a 0.45 μm filter (“Millex”, Merck). The filtered sample was diluted 50 times, and the Tyr concentration of the supernatant was determined using high-performance liquid chromatography (HPLC) (“1100 Series”, manufactured by Agilent).

The above process was conducted again after 24 hours, 48 hours, and 333 hours. The obtained results are shown in FIG. 2. In the drawing, ◯, Δ, ● and ▴ represent A (stirred, 138 mg), B (allowed to stand, 138 mg), C (stirred, 220 mg) and D (allowed to stand, 220 mg), respectively. The results show that, in the case of A and B, a decrease in supernatant concentration was not found, as the concentration of 630 mg/L was maintained over time. However, in the case of C and D, the deposit of crystals was observed, and a decrease in the supernatant Tyr concentration was confirmed. From these results, it was confirmed that the Tyr concentration in the supernatant of 630 mg/L is maintained for 333 hours, irrespective of whether it is stirred or allowed to stand as in bottles A and B.

(4) Stability Test

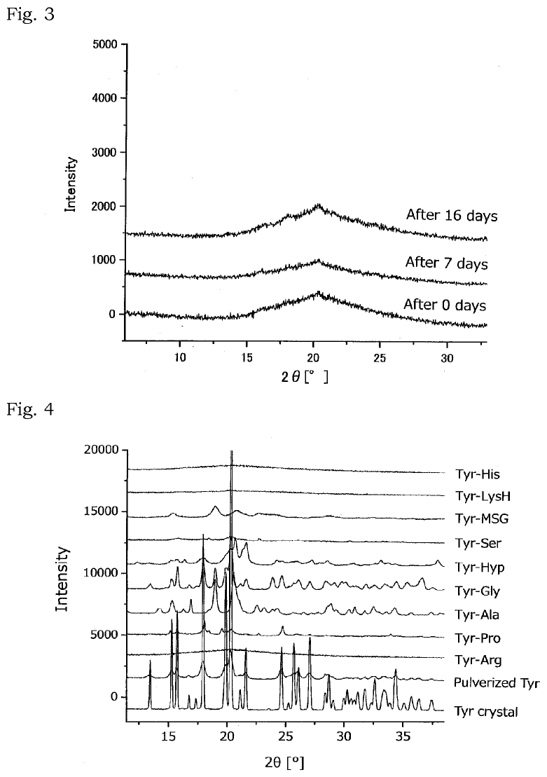

The solid obtained in (1) was placed in an aluminum pouch with silica gel, and kept in a refrigerator at 4° C. under dehumidified conditions. After 7 days and 16 days, powder X-ray diffraction patterns of the solid were measured using the powder X-ray diffraction apparatus to obtain the results shown in FIG. 3. These results show that a well-defined peak characteristic of crystal structure was not found. From these results, it was confirmed that the amorphous structure of the solid obtained in (1) was able to be maintained for 16 days.

### Examples 2-4, Comparative Examples 1-6

(1) Preparation of Tyr•Amino Acid 2 Co-Amorphous Mixture

Using the amino acid pairs as shown in Table 1, pulverization of the amino acids was carried out in a manner similar to Example 1, except that the pulverization temperature was 30° C.

TABLE 1

| | Amino Acid 1 | | Amino Acid 2 | | |
|---|---|---|---|---|---|
| | | Weight | | Weight | Forming |
| | Type | (g) | Type | (g) | Co-Amorphous |
| Comparative | Tyrosine | 10.0 | — | — | X |
| Ex. 1 | (Tyr) | | | | |
| Example 1 | Tyr | 5.1 | Arginine | 4.9 | ◯ |
| | | | (Arg) | | |
| Comparative | Tyr | 6.1 | Proline | 3.9 | X |
| Ex. 2 | | | (Pro) | | |
| Comparative | Tyr | 6.7 | Alanine | 3.3 | X |
| Ex. 3 | | | (Ala) | | |
| Comparative | Tyr | 7.1 | Glycine | 2.9 | X |
| Ex. 4 | | | (Gly) | | |
| Comparative | Tyr | 5.3 | Hydroxy- | 4.6 | X |
| Ex. 5 | | | proline | | |
| | | | (Hyp) | | |
| Example 2 | Tyr | 6.3 | Serine | 3.7 | ◯ |
| | | | (Ser) | | |
| Comparative | Tyr | 5.2 | Sodium | 4.8 | X |
| Ex. 6 | | | Glutamate | | |
| | | | (MSG) | | |
| Example 3 | Tyr | 5.0 | Lysine | 5.0 | ◯ |
| | | | Hydro- | | |
| | | | chloride | | |
| | | | (LysH) | | |
| Example 4 | Tyr | 5.4 | Histidine | 4.6 | X |
| | | | (His) | | |

(2) Confirmation of Formation of Amorphous Product

The solid obtained in (1) was measured by the same powder X-ray diffraction apparatus as Example 1, and the powder X-ray diffraction patterns of the solid are shown in FIG. 4. From the powder X-ray diffraction patterns shown in FIG. 4, it was confirmed that Tyr-Ser, Tyr-LysH, and Tyr-His formed co-amorphous products. The results are also shown in Table 1.

(3) Test to Confirm the Dissolved Amount

Into each of two 200 mL sample glass bottles (A, B), 100 mL of ultra-pure water and 130 mg of the solid from Example 3 or Tyr crystal were placed, respectively, and stirred using a magnetic stirrer (“B-1 Magnetic Stirrer Octopus”, manufactured by As One Corporation). After the powder was dissolved completely, the pH was adjusted using 5 mol/L hydrochloric acid to be within the range of 6.5-7.5. The sample bottles were immersed in a water bath (“NCB-3300”, manufactured by Tokyo Rikakikai Co., Ltd.) at 25° C., and stirred by the magnetic stirrer.

After 5 minutes, 10 minutes, 30 minutes, 1 hour, 2 hours, 24 hours, and 168 hours from the start of stirring, 2 mL was sampled from each sample bottle, and filtered using the 0.45 Ξm filter (“Millex”, Merck). The filtered sample was diluted 50 times, and the Tyr concentration of the supernatant was determined using the high-performance liquid chromatography (HPLC) (“1100 Series”, manufactured by Agilent).

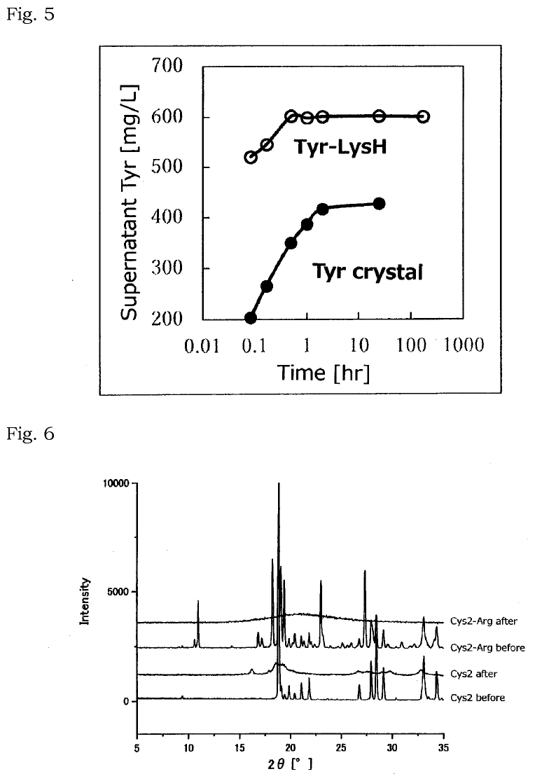

The results are shown in FIG. 5. It was confirmed that the concentration of the product from Example 3 obtained in (1) was 600 mg/L after 30 minutes, and this concentration was maintained for 168 hours, whereas the concentration of the Tyr crystal was 430 mg/L after 24 hours.

### Example 5, Comparative Example 7

(1) Preparation of Cys2•Arg Co-Amorphous Mixture

Using 6.76 g (0.0282 mol) of cystine (Cys2) (manufactured by Ajinomoto Co., Inc.) and 3.24 g (0.0186 mol) of arginine (Arg) (manufactured by Ajinomoto Co., Inc.), pulverization of the amino acids was carried out in a manner similar to Example 1, except that the pulverization temperature was 30° C. In addition, using 10 g of the same cystine, the amino acid was also pulverized similarly.

(2) Confirmation of Formation of Amorphous Product

The solid obtained in (1) was measured by the same powder X-ray diffraction apparatus as Example 1, and the powder X-ray diffraction patterns were obtained.

The results are shown in FIG. 6. In the drawing, “before” and “after” represent before pulverization and after pulverization, respectively. FIG. 6 shows that a well-defined peak characteristic of crystal structure was not found, confirming that Cys2•Arg after pulverization was amorphous. However, in the case of simplex Cys2, the crystal property remained.

### Examples 6-12

(1) Preparation of Cys2•Amino Acid 2 Co-Amorphous Mixture

Using the amino acid pairs described in Table 2, pulverization of the amino acids was carried out similar to Example 1, except that the pulverization temperature was 30° C., and the pulverizing time was 30 minutes.

TABLE 2

| | Amino Acid 1 | | Amino Acid 2 | | |
|---|---|---|---|---|---|
| | | Weight | | Weight | Forming |
| | Type | (g) | Type | (g) | Co-Amorphous |
| Comparative | Cystine | 10.0 | — | — | |
| Ex. 7 | (Cys2) | | | | |
| Example 6 | Cys2 | 6.8 | Arg | 3.2 | ◯ |
| Example 7 | Cys2 | 7.0 | Ser | 3.0 | ◯ |
| Example 8 | Cys2 | 6.2 | MSG | 3.8 | ◯ |
| Example 9 | Cys2 | 6.5 | Hypro | 3.5 | ◯ |
| Example 10 | Cys2 | 6.1 | His | 3.9 | ◯ |
| Example 11 | Cys2 | 5.7 | LysH | 4.3 | ◯ |
| Example 12 | Cys2 | 7.0 | γ-Amino- | 3.0 | ◯ |
| | | | butyric | | |
| | | | Acid | | |
| | | | (GABA) | | |

(2) Confirmation of Formation of Amorphous Product

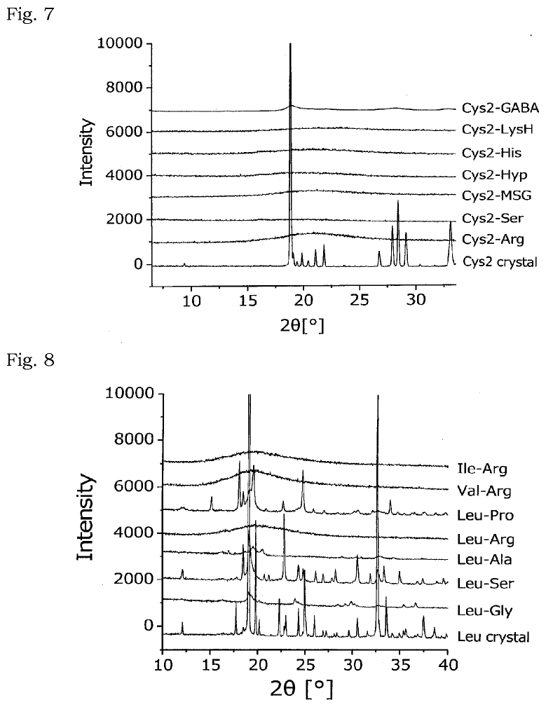

The solid obtained in (1) was measured by the same powder X-ray diffraction apparatus as Example 1, and the powder X-ray diffraction patterns of the solid are shown in FIG. 7. From the powder X-ray diffraction patterns, it was confirmed that Cys2-Arg, Cys2-Ser, Cys2-MSG, Cys2-Hypro, Cys2-His, Cys2-LysH, Cys2-GABA formed co-amorphous products. The results of the confirmation are also shown in Table 2.

### Examples 13-15, Comparative Examples 8-11

(1) Preparation of Amino Acid 1•Amino Acid 2 Co-Amorphous Mixture

Using the amino acid pairs as shown in Table 3, pulverization of the amino acids was carried out in a manner similar to Example 1, except that the pulverization temperature was 30° C., and the pulverizing time was 6 hours.

TABLE 3

| | Amino Acid 1 | | Amino Acid 2 | | |
|---|---|---|---|---|---|
| | | Weight | | Weight | Forming |
| | Type | (g) | Type | (g) | Co-Amorphous |
| Comparative | Leu | 6.4 | Gly | 3.6 | X |
| Ex. 8 | | | | | |
| Comparative | Leu | 5.6 | Ser | 4.4 | X |
| Ex. 9 | | | | | |
| Comparative | Leu | 6.0 | Ala | 4.0 | X |
| Ex. 10 | | | | | |
| Example 13 | Leu | 4.3 | Arg | 5.7 | ◯ |
| Comparative | Leu | 5.3 | Pro | 4.7 | X |
| Ex. 11 | | | | | |
| Example 14 | Valine | 4.0 | Arg | 6.0 | ◯ |
| | (Val) | | | | |
| Example 15 | Isoleucine | 4.3 | Arg | 5.7 | ◯ |
| | (Ile) | | | | |

(2) Confirmation of Formation of Amorphous Product

The solid obtained in (1) was measured by the same powder X-ray diffraction apparatus as Example 1, and the powder X-ray diffraction patterns of the solid are shown in FIG. 8. From the powder X-ray diffraction patterns, it was confirmed that Leu-Arg, Val-Arg, Ile-Arg formed co-amorphous products. These results are shown in Table 3.

### Examples 16, 17

(1) Preparation of Amino acid 1•Amino Acid 2 Co-Amorphous Mixture

Using the amino acid pairs as shown in Table 4, pulverization of the amino acids was carried out in a manner similar to Example 1, except that the pulverization temperature was 30° C.

TABLE 4

| | Amino Acid 1 | | Amino Acid 2 | | |
|---|---|---|---|---|---|
| | | Weight | | Weight | Forming |
| | Type | (g) | Type | (g) | Co-Amorphous |
| Example 16 | Methionine | 4.6 | Arg | 5.4 | ◯ |
| | (Met) | | | | |
| Example 17 | Met | 4.5 | LysH | 5.5 | ◯ |

(2) Confirmation of Formation of Amorphous Product

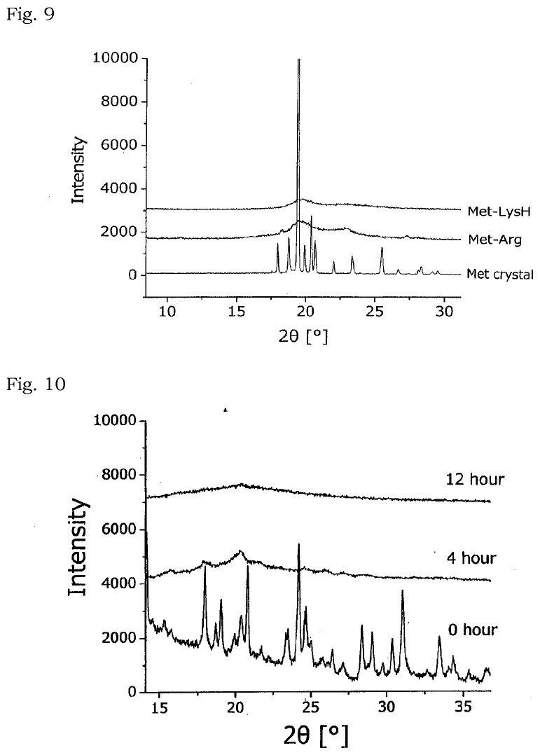

The solid obtained in (1) was measured by the same powder X-ray diffraction apparatus as Example 1, and the powder X-ray diffraction patterns of the solid are shown in FIG. 9. From the powder X-ray diffraction patterns, it was confirmed that Met-Arg and Met-Lys formed co-amorphous products. These results are shown in Table 4.

### Examples 18-21

Using tyrosine and arginine, pulverization was carried out in a manner similar to Example 1, except that the rotary speed was controlled as shown in Table 5. The confirmation results of the formation of co-amorphous products are shown in Table 5.

TABLE 5

| | | | | | Comp. | Comp. | Comp. | Comp. |
|---|---|---|---|---|---|---|---|---|
| | Ecample 18 | Example 19 | Example 20 | Example 21 | Ex. 11 | Ex. 12 | Ex. 13 | Ex. 14 |
| Rotary Speed [rpm] | 1000 | 715 | 650 | 600 | 550 | 500 | 400 | 300 |
| Forming Co-Amorphous | ◯ | ◯ | ◯ | ◯ | X | X | X | X |

### Example 22

Using tyrosine and arginine, pulverization was carried out in a manner similar to Example 1, except that the pulverizing time was controlled as shown in Table 6. The confirmation results of formation of co-amorphous products are shown in Table 6 and FIG. 10.

TABLE 6

| | Comparative | Comparative | |
|---|---|---|---|
| | Ex. 15 | Ex. 16 | Example 22 |
| Time [hr] | 0 | 4 | 12 |
| Forming Co-Amorphous | X | X | ◯ |

### Example 23: Planetary Ball Mill

20.4 g (0.11 mol) of tyrosine (Tyr) (manufactured by Ajinomoto Co., Inc.), 19.6 g (0.11 mol) of arginine (Arg) (manufactured by Ajinomoto Co., Inc.) and as the pulverizing assistant, 2 mL of ethanol were placed in a 500 mL pot made of zirconia. 456 g of 20 mm Φ balls made of zirconia were added and then placed in a planetary ball mill (“B X382”, manufactured by Kurimoto Ltd.). Nitrogen gas was also added, and grinding and mixing were carried out for 5 hours at the revolution speed of 382 rpm and a rotation speed of 842 rpm to obtain 37.7 g of co-amorphous powder. While grinding, the temperature was increased from room temperature to 60° C. at maximum.

### Example 24: Attoritor

143 g (0.79 mol) of tyrosine (Tyr) (manufactured by Ajinomoto Co., Inc.), 137 g (0.79 mol) of arginine (Arg) (manufactured by Ajinomoto Co., Inc.) and as the pulverizing assistant, 4 mL of ethanol were placed in a vessel made of stainless steel. 11.9 kg of 15 mm Φ balls made of zirconia were added in an attoritor (“MAID”, manufactured by Nippon Coke & Engineering Co., Ltd.), and also placed in the vessel. Nitrogen gas was then added to the vessel, the vessel was sealed, and grinding and mixing were carried out for 4 hours at the revolution speed of the arm of 300 rpm, while tap water was streamed into the outer periphery of the vessel, to obtain 250 g of co-amorphous material.

### Example 25: Surface Modification Apparatus (Simoloyer)

102 g (0.56 mol) of Tyrosine (Tyr) (manufactured by Ajinomoto Co., Inc.), 98 g (0.56 mol) of arginine (Arg) (manufactured by Ajinomoto Co., Inc.), and 4 mL of ethanol as the pulverizing assistant were placed in a 2 L vessel made of alumina. 2 kg of 5 mm Φ balls made of zirconia were added along with a surface modification apparatus (“CM-01”, manufactured by Zoz GmbH). Grinding and mixing were carried out for 3 hours at a revolution speed of the arm of 1000 rpm, while cooling water at 10° C. was streamed into the outer periphery of the vessel, to obtain 184 g of co-amorphous powder.

### Example 26: Spray Dryer

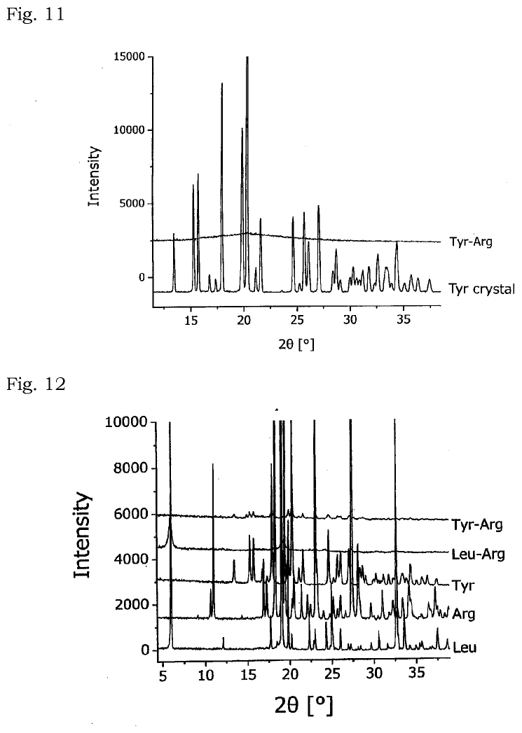

3.60 g of tyrosine and 3.46 g of arginine were dissolved in 12 L of water. Using a spray dryer (“GB210”, manufactured by Yamato Scientific Co., Ltd.), the solution of tyrosine and arginine was spray-dried at an air flow rate of 0.51 m³/min, an inlet temperature of 180-190° C., an outlet temperature of 60° C., a pressure of the atomizer gas of 0.13 MPa, and a liquid feeding rate of 20 mL/min to obtain 520 mg of powder. It was confirmed that a co-amorphous product was formed by the powder X-ray diffraction spectrum. The powder X-ray diffraction patterns are shown in FIG. 11.

### Example 27

500 mg of leucine and 664 mg of arginine were dissolved in 100 mL of water, and frozen in a freezer at −80° C. Using a lyophilizer (“FDU-1200”, manufactured by Tokyo Rikakikai Co., Ltd.), vacuum drying was conducted at a temperature of −45° C., and a vacuum pressure of 21 Pa for 4 days, to obtain 1013 mg of powder (yield: 87%). The powder X-ray diffraction patterns are shown in FIG. 12. From the powder X-ray diffraction analysis, it appears that co-amorphous material formation was improved, but did not become complete co-amorphous material.

### Example 28

300 mg of tyrosine and 288 mg of arginine were dissolved in 100 mL of water, and frozen in a freezer at −80° C. Using a lyophilizer (“FDU-1200”, manufactured by Tokyo Rikakikai Co., Ltd.), vacuum drying was conducted at a temperature of −45° C., vacuum pressure of 21 Pa for 4 days, to obtain 419 mg of powder (yield: 71%). The powder X-ray diffraction patterns are shown in FIG. 12. From the powder X-ray diffraction analysis, it appears that co-amorphous material formation was improved, but did not become complete co-amorphous material.

### INDUSTRIAL APPLICABILITY

The present invention can be utilized widely in the fields of culture medium, food, medicine, etc. where 2 or more amino acids are used, and is particularly effective dissolution of a slightly soluble amino acid is required.

We claim:

1. A composition of amino acids comprising amino acid 1 and amino acid 2, wherein at least amino acid 1 and amino acid 2 form a co-amorphous structure,
wherein said amino acid 1 is represented by R₁—CH (NH₂) COOH,
and wherein said amino acid 1 is selected from the group consisting of:
A) a basic amino acid wherein R₁ contains an amino group or imide bond,
B) a sulfur-containing amino acid wherein R₁ contains a sulfide bond, a disulfide bond, or a thiol group, and
C) tyrosine; and
wherein said amino acid 2 is represented by R₂—CH (NH₂) COOH;
wherein when the amino acid 1 is basic, then amino acid 2 is selected from the group consisting of:
a) a sulfur-containing amino acid wherein R₂ contains a sulfide bond, a disulfide bond, or a thiol group,
b) alanine,
c) valine,
e) leucine,
f) isoleucine,
g) tyrosine,
h) serine, and
i) γ-aminobutyric acid;
wherein when the amino acid 1 is the sulfur-containing amino acid, the amino acid 2 is selected from the group consisting of:
i) a basic amino acid wherein R₂ is an amino group or imide bond,
ii) alanine,
iii) valine,
iv) leucine,
v) isoleucine,
vi) tyrosine,
vii) serine,
viii) glutamic acid or a salt thereof,
ix) hydroxyproline, and
x) γ-aminobutyric acid;
wherein when the amino acid 1 is tyrosine, the amino acid 2 is serine.

2. The composition of claim 1, which consists essentially of the amino acid 1 and the amino acid 2.

3. The composition of claim 2,
wherein said amino acid 1 is selected from the group consisting of lysine, arginine, histidine, and salts thereof; and
wherein said amino acid 2 is selected from the group consisting of cystine, cysteine, methionine, alanine, valine, leucine, isoleucine, tyrosine, serine, and γ-aminobutyric acid.

4. The composition of claim 2,
wherein said amino acid 1 is cystine, cysteine, methionine, and
wherein said amino acid 2 is lysine, arginine histidine, alanine, valine, leucine, isoleucine, tyrosine, serine, glutamic acid, salts thereof, hydroxyproline, and γ-aminobutyric acid.

5. The composition of claim 3, wherein said amino acid 2 is cystine or tyrosine.

6. The composition of claim 4, wherein said amino acid 2 is serine or γ-aminobutyric acid.

7. The composition of claim 2, wherein the amino acid 1 and the amino acid 2 are selected from the group consisting of tyrosine and arginine, tyrosine and serine, tyrosine and lysine hydrochloride, tyrosine and histidine, cystine and arginine, cystine and serine, cystine and sodium glutamate monohydrate, cystine and hydroxyproline, cystine and histidine, cystine and γ-aminobutyric acid, leucine and arginine, leucine and lysine hydrochloride, leucine and histidine, leucine and cystine, isoleucine and arginine, isoleucine and lysine hydrochloride, isoleucine and histidine, isoleucine and cystine, valine and arginine, valine and lysine hydrochloride, valine and histidine, valine and cystine, methionine and arginine, and methionine and lysine hydrochloride.

8. The composition of claim 2, wherein the amino acid 1 and the amino acid 2 are selected from the group consisting of tyrosine and arginine, tyrosine and serine, tyrosine and lysine hydrochloride, tyrosine and histidine, cystine and arginine, cystine and lysine hydrochloride, cystine and serine, cystine and sodium glutamate monohydrate, cystine and hydroxyproline, cystine and histidine, cystine and γ-aminobutyric acid, leucine and arginine, isoleucine and arginine, valine and arginine, methionine and arginine, and methionine and lysine hydrochloride

9. The composition of claim 2, wherein the amino acid 1 is tyrosine, and the amino acid 2 is lysine hydrochloride, serine, or histidine.

10. The composition of claim 2, wherein the amino acid 1 is cystine, and the amino acid 2 is serine, lysine hydrochloride, or γ-aminobutyric acid.

11. The composition of claim 2, wherein the amino acid 1 is arginine, and the amino acid 2 is valine, leucine, or isoleucine.

12. The composition of claim 2, wherein the amino acid 1 is lysine hydrochloride, and the amino acid 2 is tyrosine, cystine or methionine.

13. The composition of claim 1, wherein the ratio of amino acid 1 to amino acid 2 in the composition is a molar ratio of 0.1 to 10.

14. The composition of claim 13, wherein the ratio of amino acid 1 to amino acid 2 is a molar ratio of 6:4 to 4:4.

15. A beverage containing the composition of claim 1.

16. A medicine containing the composition of claim 1.

17. An aromatic or cosmetic containing the composition of claim 1.

18. A method of producing the composition of claim 1, which comprises
a) placing at least two amino acids into a ball mill at a molar ratio of 1:0.1-10, and adding a pulverizing assistant at 0.1 to 5 weight % relative to the total amount of all amino acids, and
b) driving the ball mill at a rotational speed of 200 to 1200 rpm for 15 minutes to 12 hours.

19. The method of claim 18, wherein the ball mill is cooled to 5 to 15° C. during the driving of the ball mill.
